(19) United States
(12) Patent Application Publication
(10) Pub. No.: US 2005/0042677 A1
(43) Pub. Date: Feb. 24, 2005

(54) Generation of compound libraries utilizing molecular imprints including a double or anti-idiotypic approach

(76) Inventors: Klaus Mosbach, Furulund (SE); Lei Ye, Lund (SE)

Correspondence Address: BURNS DOANE SWECKER & MATHIS L L P, POST OFFICE BOX 1404, ALEXANDRIA, VA, 22313-1404, US

(21) Appl. No.: 10/490,632

(22) PCT Filed: Sep. 27, 2002
(86) PCT No.: PCT/SE02/01770

(30) Foreign Application Priority Data
Sep. 28, 2001 (SE) 0103249-0

(51) Int. Cl. G01N 33/53; G01N 33/543

(52) U.S. Cl. 435/7.1; 436/518; 525/54.1

(57) ABSTRACT

The present invention relates to a method of producing new chemical entities comprising the steps of: (i) taking a chemical entity as the template to prepare a molecularly imprinted polymer (MIP), (ii) removing the template from the MIP, (iii) using the specific binding sites of the MIP to direct, or facilitate, the syntheses of new chemical entities for the generation of compound libraries using molecularly imprinted polymers, and to a use of such compound libraries.

### TECHNICAL FIELD OF THE INVENTION

The present invention relates to a method of producing new chemical entities comprising the steps of:
(i) taking a chemical entity as the template to prepare a molecularly imprinted polymer (MIP),
(ii) removing the template form the MIP,
(iii) using the specific binding sites of the MIP to direct, or facilitate, the synthesis of new chemical entities,
for the generation of compound libraries using molecularly imprinted polymers, and to a use of such compound libraries.

### BACKGROUND OF THE INVENTION

In the process of drug development, researchers invest much effort in creating lead compounds displaying bioactivity against identified targets. This has been realized by both the rational design¹ and the combinatorial methodology,² or a combination of both.³ Rational drug design requires that the target be well characterized, i.e. a detailed three-dimensional structure of the target must be available to the medicinal chemists. In combinatorial chemistry, large amounts of compounds are synthesized and subjected to high through screening, in order to find a handful hit molecules. It is possible to directly utilize certain binding groups on the surface of a protein, to generate strong affinity ligands capable of selectively binding the same biomacromolecule.⁴ Similarly, compounds that possess a complementary structure to a target biomolecule, such as enzymes and receptors, can be directly synthesized using the active site of the target as a reaction mould. ⁵ However, this “target directed synthesis” method is applicable only when the target has been isolated and its structure known, because the choice of reactants relies on the types of functional groups present in its active site. Utilizable synthetic reactions are also limited due to the presence of various side chain functional groups of the target, which are reactive under the physiological conditions.

It is often the case that the three-dimensional structure of a biological target is unresolved, instead, its inhibitor/agonist/antagonist is known. ⁶ Under this circumstance, the present invention can be used to first prepare a molecularly imprinted polymer (MIP) having binding site that mimics the biomolecule's active center. The binding site of the MIP is then used as a reaction mold to direct the synthesis of newt inhibitiors/agonists/antagonists.

By molecular imprinting, co-polymerization of functional monomers and cross-linking monomers is carried out in the presence of a molecular template, which results in a rigid polymer matrix embedding the template. Removal of the template reveals binding sites specific to the template or its close analogue. Molecularly imprinted polymers are much more stable than biological receptors, and much easier to produce. They have great potential to replace, or supplement biological receptors in all affinity related applications.

In molecular imprinting, the assembly of template-functional monomer complex prior to and during the polymerization reaction, as well as re-binding of the template by the obtained polymer is driven by various molecular interactions between the template and the functional monomers. Wulff and Poll described a method of using reversible covalent bond for molecular imprinting of an optically active compound, as well as use of the polymer for separating an optically active antipode from a racemate mixture (Wulff, G.; Poll, H. G. Makromol. Chem. 1987, 188, 741-748). U.S. Pat. No. 5,310,648 describes use of metal chelating functional monomers for preparing an imprinted polymer matrix. More favorably, non-covalent interactions have been used in PCT applications WO 93/09075 and WO 98/07671 for preparing a chiral solid-phase chromatography material containing molecular imprints of an optically pure enantiomer to be separated.

PCT application WO 99/33768 describes use of molecularly imprinted polymers as artificial receptors in the screening of combinatorial libraries.

PCT application WO 95/21673 describes preparation and application of artificial anti-idiotypic antibodies obtained by molecular imprinting, in which a molecularly imprinted polymer is used as a mold in a subsequent polymerization step to give a new polymeric affinity material.

In this invention, we use in the first step a known bioactive molecule, such as an enzyme inhibitor, a receptor agonist or antagonist, or an affinity ligand, as the template to prepare a molecularly imprinted polymer (the primary MIP). Following removal of the template, the specific binding site of the primary MIP is used to direct the synthesis of new compounds having functionalities and shapes that are complementary to the binding cavity of the primary MIP. A focused compound library can be generated, which contains close analogues of the original inhibitor, agonist/antagonist or affinity ligand, which accordingly display similar bioactivities.

### SUMMARY OF THE INVENTION

The object of the invention is to provide a method of producing new chemical entities wherein the above mentioned drawbacks have been eliminated or alleviated.

According to the present invention this object is achieved by a method of producing new chemical-entities comprising the steps of:
(i) taking a chemical entity as the template to prepare a molecularly imprinted polymer (MIP),
(ii) removing the template form the MIP,
(iii) using the specific binding sites of the MIP to direct, or facilitate, the synthesis of new chemical entities,
wherein a focused compound library is generated by screening more than one reactant and wherein hit products are those that are obtainable only via the site directed synthesis provided by the imprinted polymer.

A further object of the present invention is to provide a use of the hit products according to any one of claims 1 and 7-10 for iterative lead optimisation.

According to the present invention this object is achieved by choosing a new template from one of the hit products for the preparation of a new MIP, which subsequently is used to generate a new focused compound library.

Other distinguishing features and advantages of the invention will appear from the following specification and the appended claims.

### BRIEF DESCRIPTION OF THE DRAWINGS

Preferred embodiments of the present invention will now be described in more detail, reference being made to the enclosed drawings, in which:

FIG. 1 schematically shows the use of a molecularly imprinted polymer to generate new compounds. After removal of the template (shaded) from the MIP, the specific binding site is used to direct the assembly of the reactants to give new products.

FIG. 2 shows the structures of the reactants and products described in the present invention.

FIG. 3 shows binding of the template (1) by the imprinted polymer. (solid circle) and the non-imprinted polymer (open circle) in Example -b 3.

FIG. 4 shows the site-directed re-synthesis of the original template (1) using the imprinted polymer (solid circle) in Example 4. The non-imprinted polymer is used as a control (open circle) under the same condition.

### DETAILED DESCRIPTION OF PREFERRED EMBODIMENTS

The molecular imprinting approach according to the invention comprises the steps of: 1) Preparation of a molecularly imprinted polymer using a known bioactive molecule as the template; 2) Removing the template from the polymer matrix to leave specific binding sites; 3) Using the specific binding sites as a reaction mold to synthesize new compounds.

The specific binding sites of the imprinted polymer are obtainable by polymerizing functional monomers and, optionally, cross-linking monomers, in the presence of a template molecule, whereby non-covalent or covalent interactions are formed between said functional monomers and said template molecule, and removing said template from the molecularly imprinted polymer. The specific binding sites are utilized to selectively bind appropriate reactants, which react to form chemical products.

In the invention the template is a known bioactive molecule, for example an enzyme inhibitor, an agonist or antagonist, or an affinity ligand. The obtained imprinted polymer is accordingly a mimic of the target biomolecule, or more appropriately, the imprinted polymer contains binding sites that mimic the active center of the target biomolecule.

In the present invention the term polymer covers both organic and inorganic polymers. Examples of organic polymers are those based on polyacrylate, polystyrene, polyanaline and polyurethane. In one aspect said polymers may be cross-linked to various extents. The polymers are obtainable by conventional polymerization reactions for example free radical polymerization or condensation polymerization. An example of inorganic polymer is a silica gel obtained by hydrolysis of precursor monomers e.g. alkoxysilanes that are commonly used for preparing silica particles.

The molecularly imprinted polymers in the present invention are synthesized in various configurations including monoliths, irregular particles, microspheres, membranes, films, and monolayers. The imprinted polymers are also in situ synthesized in microtitre plate wells.

In one example the molecularly imprinted polymer is synthesized in the form of a monolith, which is ground to particles with appropriate sizes, optionally of 10-25 μm.

In another example the imprinted polymer is in situ synthesized in microtitre plate wells or on microchips. The polymers may be in the form of continuous films or separate spots.

In the present invention the molecularly imprinted polymer is used to direct the synthesis of new chemical entities, typically compounds potentially useful as enzyme inhibitors, agonists or antagonists, or affinity ligands.

The imprinted polymer is used to generate a focused compound library by introducing different reactants to the polymer's specific sites. The synthetic reactions may be carried out individually, or in parallel. By parallel reaction it means different products are synthesized simultaneously with the imprinted polymer in one pot. In the case of parallel synthesis, the obtained products are analyzed to find out respective reactants.

To identify a real site-directed synthesis with the imprinted polymer, a non-imprinted polymer is used as a control. The hit products (reactants) are those obtained only with the imprinted polymer, while product yields with the non-imprinted polymer are used as the background values.

The new compounds obtained by the site-directed synthesis can be separated from the imprinted polymer and directly used in bioassays. Alternatively, the reactants identified to give the desired new compounds are used in the scale up synthesis for the corresponding products, which are used in further investigations.

The new, compounds obtained by the present invention are potentially useful as enzyme inhibitors, agonists or antagonists, or as affinity ligands.

### EXAMPLES

Synthesis of a Molecularly Imprinted Polymer

A molecularly imprinted polymer is prepared using a kallikrein inhibitor (1) as the template. The obtained imprinted polymer contains specific binding site that mimics the active center of the protease tissue kallikrein.

### Example 1

### Preparation of the Molecularly Imprinted Polymer

The kallikrein inhibitor (1) is synthesized according to a literature method.

The inhibitor (1) is dissolved in N,N-dimethylformamide (DMF) and treated with an anion exchange resin, Amberlite IRA-400 from Fluka (Dorset, UK). Removal-of solvent gives 1 in the free base form. The free base (0.3 mmol), (2-trifluoromethyl)acrylic acid (2.4 mmol), di-vinylbenzene (12 mmol) and azobisisobutyronitrile (0.12 mmol) were dissolved in DMF (2 mL). The solution is saturated with dry nitrogen, followed by polymerization at 60° C. for 16 h. The polymer monolith is ground and fractionated to give appropriately sized particles (10-25 μm). The template is removed by repetitive washing in methanol:acetic acid (90:10, v/v), until no template car, be detected in the washing solvent using a UV spectro-meter. A non-imprinted polymer is prepared in the same way except omission of the template.

### Example 2

### Chromatographic Evaluation of the Imprinting Effect

Polymer particles are slurry packed into standard HPLC columns (250×4.6 mm) using an air driven fluid pump. A LaChrom L-7100 solvent delivery system, a L-7455 diode array detector and a software package D-7000 HPLC System Manager (Merck KgaA, Darmstadt, Germany) are used for the chromatographic analyses. The test compounds (20 μL at 1.0 mg/mL) are loaded onto both the imprinted and the non-imprinted columns, which are eluted applying a gradient of 1-10% acetic acid in acetonitrile (1.0 mL/min) within 30 min. Acetone is used as the void marker. Capacity factor (k′) is calculated as (t−t₀)/t₀, where t is the retention time of the test compound and t₀ of the void marker. The normalized retention index (RI) is calculated as:
RI(%)=[k′_{analyte}(MIP)/k′_{analyte}(control)]/[k′ₜₑₘₚₗₐₜₑ(MIP)/k′ₜₑₘₚₗₐₜₑ(control)]×100

where k′_{analyte} (MIP) and k′ₜₑₘₚₗₐₜₑ(control) are the capacity factors of an analyte on the MIP column, and of 1 on the control column respectively. By definition, the retention index is a measure of the relative specific retention of an analyte-on the MIP column, giving a value of 100% for the template compound.

TABLE 1
Chromatographic evaluation of the imprinting
effect

| | Capacitor factor | | |
|---|---|---|---|
| | (k′) | | Retention index |
| Test compounds | MIP | Control | (RI) |
| 1 | 12.5 | 9.0 | 100 |
| 2-(4-amidinophenylamino)- | 7.0 | 6.2 | 81 |
| 4,6-dichloro- | | | |
| s-triazine | | | |
| 4-Aminobenzamidine | 2.6 | 3.6 | 52 |
| dihydrochloride | | | |
| Cyanuric chloride | 0 | 0 | — |

### Example 3

### Batch Mode Binding Analysis

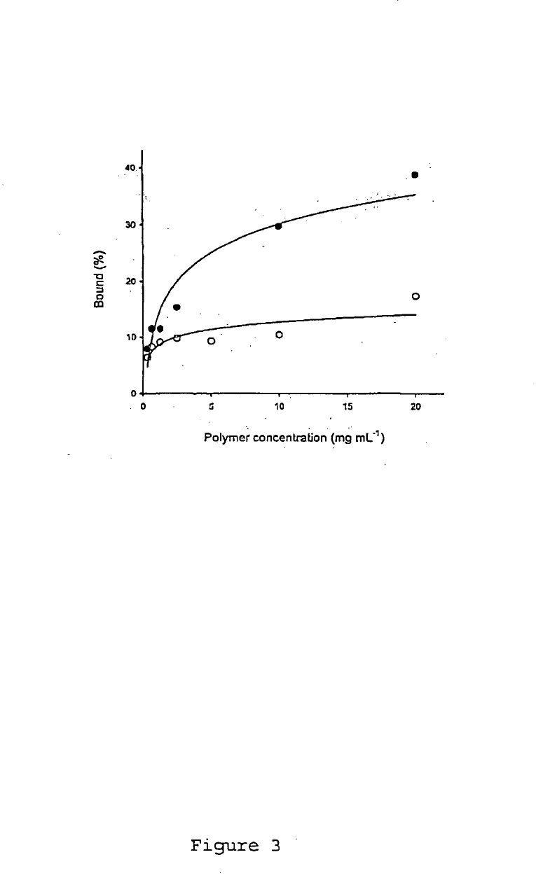

Template 1 (100 μg) is incubated with increasing amount of the imprinted and the non-imprinted polymer in DMF (1.0 mL) at 20° C. for 16 h. Polymer particles are removed by centrifugation, the supernatant is analyzed with reverse phase HPLC. A Chromolith Performance column (RP-18e) from Merck (Darmstadt, Germany) is used with a gradient elution: 0-10 min, 20-50% acetonitrile in water, both containing 0.1% trifluoroacetic acid at a flow rate of 1 mL min⁻¹. The amount of 1 binds to the polymer is calculated by subtraction of the free from the total amount added using an established calibration curve. The result is shown in FIG. 3.

Generation of New Compounds Using the Imprinted Binding Sites

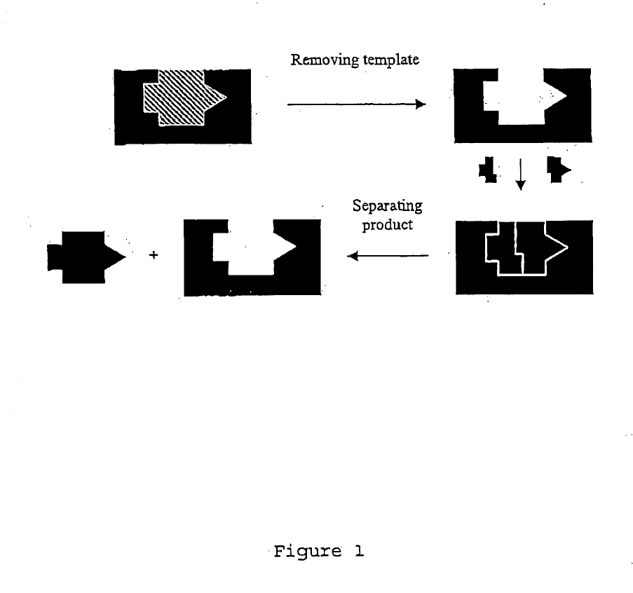

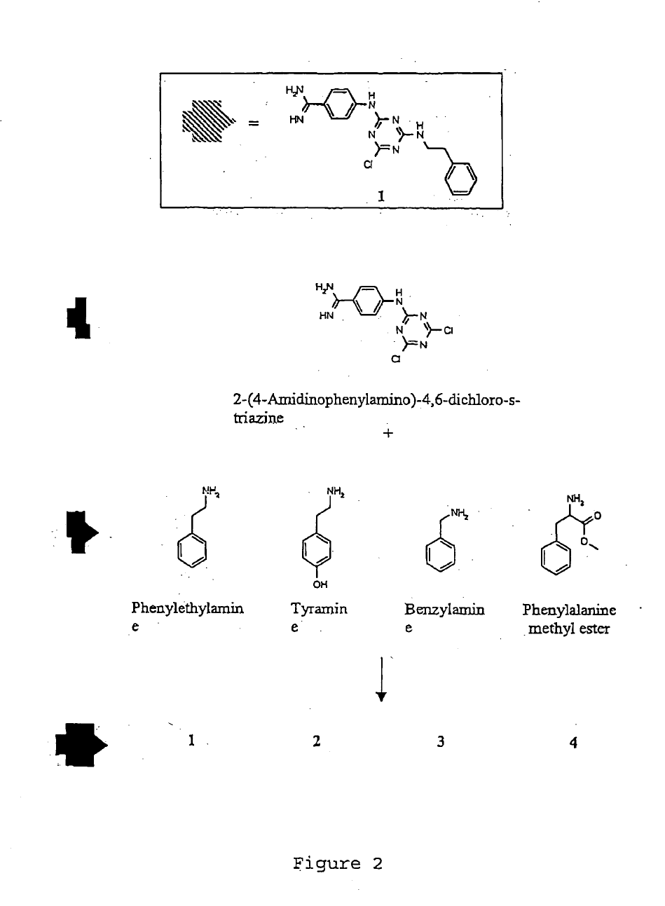

The molecularly imprinted polymer is used for the site-directed synthesis of new compounds (FIGS. 1 and 2). Because the MIP mimics the enzyme kallikrein, use of the artificial active site in the MIP is expected to result in new kallikrein inhibitors.

### Example 4

### Re-Synthesis of 1 using the Molecularly Imprinted Polymer

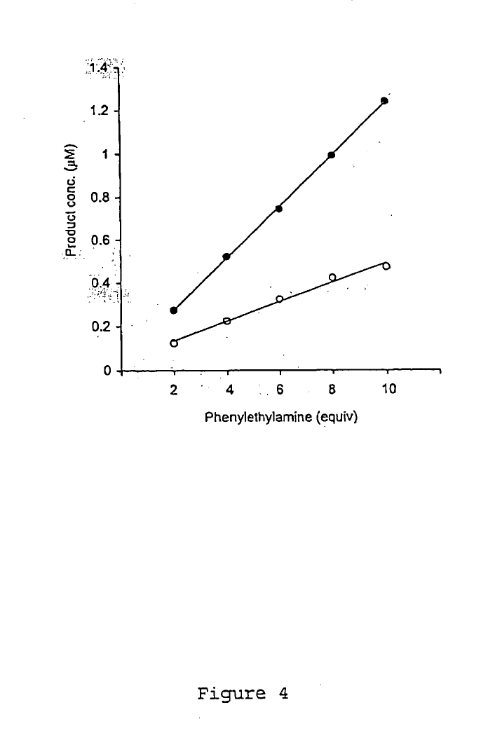

2-(4-Amidinophenylamino)-4,6-dichloro-s-triazine (10 μg, 31.3 nmol) is incubated with the imprinted and the control polymer (10 mg) in DMF (600 μL) at 20° C. for 2 h. Different amount of phenylethylamine dissolved in DMF (100 μL) is then added, and the reaction continued at 20° C. on a rocking table that provides gentle mixing. After 8 h reaction, acetic acid (100 μL) is added and the mixture further incubated at 20° C. for another hour. Polymer particles are removed using Centrifugal Microsep Devices (MWCO 300K) from PALL Gelman Laboratory (Ann Arbor, Mich., USA). The filtrate is directly analyzed by reverse phase HPLC. Synthetic result is shown in FIG. 4.

### Example 5

### Site-Directed Synthesis of 2, 3, and 4

Synthesis of new compounds using the imprinted binding site is attempted. To the imprinted polymer are feed reactants leading to products 2, 3 and 4. At the low concentration level, none of the products can be obtained in free solution. If the MIP can facilitate synthesis of a specific product in comparison with the non-imprinted polymer, the product can be considered as a potential kallikrein inhibitor.

2-(4-Amidinophenylamino)-4,6-dichloro-s-triazine (31.3 mmol) is incubated with the MIP (10 mg) in DMF (600 μL) at 20° C. for 2 h. Different amine reactants (10 equiv) in 100 μL of DCM are then added, and the reactions continued for 8 h. After the reaction, acetic acid (100 μL) is added, and the mixture is incubated for another hour. Polymer particles are removed by centrifugal filtration. Product content in the filtrate is quantified by HPLC analysis. A Chromolith Performance column (RP-18e) from Merck (Darmstadt, Germany) is used with a gradient elution: 0-10 min, 20-50% acetonitrile in water, both containing 0.1% trifluoroacetic acid at a flow rate of 1 mL min⁻¹. Relative yields of 2, 3 and 4 are normalized to that of 1. None of compound 2, 3 and 4 can be obtained when the synthesis is carried out-using the non-imprinted polymer. The result of the site-directed synthesis with the imprinted polymer is shown in Table 2.

TABLE 2
MIP-assisted synthesis of kallikrein inhibitors

| | Retention time | Prod. conc. | |
|---|---|---|---|
| Product | (min) | (μM) | Relative yield (%) |
| 1 | 8.4 | 1.51 | 100 |
| 2 | 6.5 | 0.31 | 21 |
| 3 | 6.6 | 0.52 | 34 |
| 4 | 8.5 | 0 | 0 |

### Example 6

### Site-Directed Synthesis of Multiple Products

2-(4-Amidinophenylamino)-4,6-dichloro-s-triazine (10 μg, 31.3 nmol) is incubated with the imprinted polymer (10 mg) in DMF (600 μL) at 20° C. for 2 h. Tyramine (leading to 2, 10 equiv) or benzylamine (leading to 3, 10 equiv) is mixed with phenylethylamine (leading to 1, 10 equiv) in DMF (100 μL), and the solution added into the MIP suspension. The reaction continues at 20° C. on a rocking table that provides gentle mixing. After 8 h reaction, acetic acid (100 μL) is added and the mixture further incubated at 20° C. for another hour. Polymer particles are removed using Centrifugal Microsep Devices (MWCO 300K) from PALL Gelman Laboratory (Ann Arbor, MI, USA). The filtrate is directly analyzed by reverse phase HPLC to calculate the yield of 1, 2 and 3. Table 3 lists the result of the site-directed parallel synthesis of the new products. The relative yields are normalized to that of 1 obtained in Example 5.

TABLE 3
Site-directed parallel synthesis of multiple
products

| | | | Relative |
|---|---|---|---|
| Reactants | | Product | yield (%) |
| 2-(4-Amidinophenylamino)- | Phenylethylamine | 1 | 82 |
| 4,6-dichloro- | Tyramine | 2 | 11 |
| s-triazine | | | |
| 2-(4- | Phenylethylamine | 1 | 65 |
| Amidinophenylamino)- | Benzylamine | 3 | 17 |
| 4,6-dichloro-s- | | | |
| triazine | | | |

Scale Up Synthesis of the Hit Products

The products 2 and 3 are identified as the hit products, since these are successfully obtained only by the MIP-directed synthesis. For further investigation, scale up synthesis is carried out.

### Example 7

### Scale up Synthesis of 2 and 3

Compound 2 is synthesized according to a literature method. ⁷

For the synthesis of 3, 2-(4-amidinophenylamino)-4,6-dichloro-s-triazine (473 mg, 1.5 mmol) is dissolved in DMF (15 mL). Benzylamine (164 μL, 1.5 mmol) in DMF (7.5 mL) is added. The mixture is stirred at 20-30° C. for 48 h. After the reaction is completed, solvent is removed by rotary evaporation. The residue is washed with water (2×30 mL) and centrifuged to remove supernatant, and then dried in vacuum. The crude product is purified by silica column chromatography using chloroform:methanol:acetic acid (8/4/0.5, v/v). Yield: 58%. ¹H NMR (400 MHz, DMSO-d₆): δ(ppm) 10.50 (s, 1H, NH), 9.25 (s, 1H, NH), 8.90 (bs, 2H, NH), 8.02 (m, 1H), 7.80 (m, 3H), 7.4-7.15 (m, 5H, Ph), 4.78 (bd, 1H, NH), 4.55 (s, 2H, CH₂).

Evaluation of Bioactivity

The identified new compounds 2 and 3 are subjected to enzyme inhibition tests.

### Example 8

### Determination of Inhibition Constants (Kᵢ) for Tissue Kallikrein

Inhibition constants of compounds 1, 2 and 3 for tissue kallikrein are determined according to the literature method described by Burton and Lowe. ⁷ The results are listed in Table 4. As seen the new compounds obtained by the site-directed synthesis displays the expected bioactivity, while 3 shows approximately the same inhibition efficacy as that of the original template (1).

TABLE 4
Inhibition constants for tissue kallikrein

| Compound | Kᵢ (μM) |
|---|---|
| 2-(4-Amidinophenylamino)-4,6- | >100 |
| dichloro-s-triazine | |
| 1 | 4.5 |
| 2 | 40 |
| 3 | 5.2 |

### REFERENCES

1. Briesewitz, R; Ray, G. T.; Wandless, T. J.; Crabtree, G. R. Affinity modulation of small-molecule ligands by borrowing endogenous protein surfaces. Proc. Natl. Acad. Sci. USA 1999, 96, 1953-1958.
2. Terrett, N. K.; Gardner, M.; Gordon, D. W.; Kobylecki, R. J.; Steele, J. Drug discovery by combinatorial chemistry—the development of a novel method for the rapid synthesis of single compounds. Chem. Eur. J. 1997, 3, 1917-1920.
3. Kramer, R. H.; Karpen, J. W. Spanning binding sites on allosteric proteins with polymer-linked ligand dimers. Nature 1998, 395, 710-713.
4. Kempe, M.; Glad, M.; Mosbach, K. An approach towards surface imprinting using the enzyme ribonuclease A. J. Mol. Recogn. 1995, 8, 35-39.
5. U.S. Pat. No. 6,127,154.
6. Kubinyi, H. Chances favors the prepared mind—from serendipity to rational drug design. J. Recept. Signal Transduction Res. 1999, 19, 15-39.
7. Burton, N. P.; Lowe, C. R. Design of novel affinity adsorbents for the purification of trypsin-like proteases. J. Mol. Recogn. 1992, 5, 55-68.

1. A method of producing new chemical entities comprising the steps of:
(i) taking a chemical entity as the template to prepare a molecularly imprinted polymer (MIP),
(ii) removing the template from the MIP, and
(iii) using the specific binding sites of the MIP to direct, or facilitate, the synthesis of new chemical entities,
wherein a focused compound library is generated by screening more than one reactant and wherein hit products are those that are obtainable only via the site directed synthesis provided by the imprinted polymer.

2. A method according to claim 1, wherein the chemical entity that is chosen as the template has desired physical, chemical, biochemical or physiological properties.

3. A method according to claim 1, wherein the chemical entity that is chosen as the template is an enzyme inhibitor, an agonist or antagonist, or an affinity ligand.

4. A method according to claim 1, wherein the imprinted polymer is an organic polymer or an inorganic polymer.

5. A method according to claim 1, wherein the new chemical entities produced are the same as, or different from, the original template.

6. A method according to claim 1, wherein the syntheses of the new chemical entities are carried out by organic reactions.

7. A method according to claim 1, wherein the syntheses of the new chemical entities are carried out by polymerization reactions.

8. A method according to claim 1, wherein the syntheses of the new chemical entities is are carried out separately.

9. A method according to claim 1, wherein the syntheses of several new chemical entities are carried out simultaneously in one pot.

10. A method according to claim 1, wherein the hit products that are identified are subjected to scale up synthesis.

11. A method for iterative lead optimization, wherein a new template is chosen from one of the hit products of claim 1 for the preparation of a new MIP, which subsequently is used to generate a new focused compound library.

12. A method according to claim 1, comprising the further step of using said hit products to replace the original template.

13. (Canceled)

14. A method according to claim 2, wherein the chemical entity that is chosen as the template is an enzyme inhibitor, an agonist or antagonist, or an affinity ligand.

15. A method for iterative lead optimization, wherein a new template is chosen from one of the hit products of claim 7 for the preparation of a new MIP, which subsequently is used to generate a new focused compound library.

16. A method for iterative lead optimization, wherein a new template is chosen from one of the hit products of claim 8 for the preparation of a new MIP, which subsequently is used to generate a new focused compound library.

17. A method for iterative lead optimization, wherein a new template is chosen from one of the hit products of claim 9 for the preparation of a new MIP, which subsequently is used to generate a new focused compound library.

18. A method for iterative lead optimization, wherein a new template is chosen from one of the hit products of claim 10 for the preparation of a new MIP, which subsequently is used to generate a new focused compound library.

19. A method according to claim 8, comprising the further step of using said hit products to replace the original template.

20. A method according to claim 9, comprising the further step of using said hit products to replace the original template.

21. A method according to claim 10, comprising the further step of using said hit products to replace the original template.

22. A method according to claim 11, comprising the further step of using said hit products to replace the original template.
